SEQUENCE LISTING (20 sequences)

SEQ ID NO: 1 - DNA, 34 bp, Artificial Sequence
  LoxP site
ataacttcgtataatgtatgctatacgaagttat

SEQ ID NO: 2 - DNA, 34 bp, Artificial Sequence
  Lox511 site
ataacttcgtataatgtatactatacgaagttat

SEQ ID NO: 3 - DNA, 34 bp, Artificial Sequence
  Lox66 site
ataacttcgtataatgtatgctatacgaacggta

SEQ ID NO: 4 - DNA, 34 bp, Artificial Sequence
  Lox71 site
taccgttcgtataatgtatgctatacgaagttat

SEQ ID NO: 5 - DNA, 34 bp, Artificial Sequence
  Lox5171 site
ataacttcgtataatgtgtactatacgaagttat

SEQ ID NO: 6 - DNA, 34 bp, Artificial Sequence
  Lox2272 site
ataacttcgtataaagtatcctatacgaagttat

SEQ ID NO: 7 - DNA, 34 bp, Artificial Sequence
  m2 site
ataacttcgtataagaaaccatatacgaagttat

SEQ ID NO: 8 - DNA, 34 bp, Artificial Sequence
  m3 site
ataacttcgtatataataccatatacgaagttat

SEQ ID NO: 9 - DNA, 34 bp, Artificial Sequence
  m7 site
ataacttcgtataagatagaatatacgaagttat

SEQ ID NO: 10 - DNA, 34 bp, Artificial Sequence
  m11 site
ataacttcgtatacgataccatatacgaagttat

SEQ ID NO: 11 - DNA, 34 bp, Artificial Sequence
  FRT site
gaagttcctatactttctagagaataggaacttc

SEQ ID NO: 12 - DNA, 34 bp, Artificial Sequence
  FRT G site
gaagttcctatactctctggagaataggaacttc

SEQ ID NO: 13 - DNA, 34 bp, Artificial Sequence
  FRT H site
gaagttcctatactatcttgagaataggaacttc

SEQ ID NO: 14 - DNA, 34 bp, Artificial Sequence
  FRT F3 site
gaagttcctatactatttggagaataggaacttc

SEQ ID NO: 15 - DNA, 20 bp, Artificial Sequence
  KIF2A-E10F primer
ccttcgatgactcagctcct

SEQ ID NO: 16 - DNA, 20 bp, Artificial Sequence
  KIF2A-EnormalF primer
aatatttgaaaggggcatgg

SEQ ID NO: 17 - DNA, 20 bp, Artificial Sequence
  KIF2A-EnormalR primer
ttttcccacttccagtctgc

SEQ ID NO: 18 - DNA, 23 bp, Artificial Sequence
  KIF2A-E13R primer
agtaagtcaaacacctttccact

SEQ ID NO: 19 - DNA, 20 bp, Artificial Sequence
  KIF2A-EdegenererF primer
gttggtcgagaccatcttcg

SEQ ID NO: 20 - DNA, 19 bp, Artificial Sequence
  KIF2A-EdegenererR primer
ttgtccgtacgcgaaacag

(19) United States
(12) Patent Application Publication
(10) Pub. No.: US 2020/0332278 A1
(43) Pub. Date: Oct. 22, 2020

(54) METHODS TO GENERATE CONDITIONAL KNOCK-IN MODELS

(71) Applicants: UNIVERSITE DE STRASBOURG, STRASBOURG (FR); INSERM (INSTITUT NATIONAL DE LA SANTÉ ET DE LA RECHERCHE MÉDICALE), PARIS (FR); CENTRE NATIONAL DE LA RECHERCHE SCIENTIFIQUE, PARIS (FR)

(72) Inventors: MARIE-CHRISTINE BIRLING-VILLA, ILLKIRCH (FR); JAMEL CHELLY, STRASBOURG (FR)

(21) Appl. No.: 16/621,706

(22) PCT Filed: Jun. 15, 2018
(86) PCT No.: PCT/EP2018/066015

§ 371 Date: Dec. 12, 2019

(30) Foreign Application Priority Data
Jun. 16, 2017 (EP) 17305741.5

(51) Int. Cl. C12N 15/10; C12N 15/11; C12N 15/85; A01K 67/027

(52) U.S. Cl. CPC C12N 15/102; C12N 15/11; C12N 15/85; A01K 2217/072; A01K 2227/105; A01K 2217/052; A01K 67/0278

(57) ABSTRACT

The present invention relates to an innovative strategy to generate conditional point mutation models using the FLEx switch system. The approach offers the possibility of creating a conditional knock-in model with the desired mutation at any position in the gene and at any time.

### FIELD OF THE INVENTION

The present invention relates to methods and compositions for generating conditional knock-in alleles.

### BACKGROUND OF THE INVENTION

Spatial- and temporal-restricted knock-out mouse models can be generated using Cre/lox recombination system. However, expression of a point mutation, irrespective of its position in a given gene, in a tissue- and time-restricted manner is still a challenging issue and efficient strategies remain lacking.

Though constitutive knock-in mouse models are a straightforward and widely used approach, investigations of conditional knock-in models are also required because very often constitutive knock-in and/or knock-out models are not viable. Moreover, for an increasing number of genes human phenotypes associated with the missense mutations are different from those associated with the loss of function mutations.

Until now and thanks to constructions using LoxP sites flanking the normal version of the exon of interest followed by its mutated version, it was only possible to generate conditional knock-in models corresponding to mutations located in the last exon(s) of genes of interest (Scekic-Zahirovic et al., EMBO J. 2016 May 17; 35(10): 1077-1097). In this configuration, the Cre-mediated recombination between two directly repeated LoxP sites leads to an irreversible excision of the intervening sequence, i.e. the normal version of the exon(s), and therefore to an expression of the allele with the point mutation.

Schnütgen et al. (Nat Biotechnol. 2003 May; 21(5):562-5) disclosed a Cre-dependent genetic switch (FLEx switch) using the capacity of the Cre-recombinase to invert or excise DNA fragment depending on the orientation of the lox sites and the availability of both wild-type and mutant lox sites. As a result, the expression of a given allele was turned off, while the expression of another one was concomitantly turned on. More specifically, to generate the mouse model, the plasmid construct contained one pair of wild type loxP sites flanking a head-to-head oriented sequences of interest, and one pair of modified lox511 sites flanking the inverted sequence of interest and a selection cassette, with an alternate organization and a head-to-head orientation within each pair of sites.

The FLEx switch system was shown to be efficient and functional as far as the inverted sequences are different (i.e., eGFP coding sequence in the orientation 5′-3′ and LacZ coding sequence in the orientation 3′-5′) (Schnütgen et al., supra). However, attempts using this FLEx switch system to generate conditional point mutation models were unsuccessful and mimicked constitutive knock-out models.

Thus, the need of a method for generating conditional knock-in models based on the FLEx switch system remains unsatisfied.

### SUMMARY OF THE INVENTION

The inventors herein developed a FLEx switch system that can be used to generate conditional point mutation models. This approach offers the possibility of creating a conditional knock-in model with the desired mutation at any position in the gene and at any time. This strategy offers also the possibility to develop point mutation models and to assess phenotype reversibility in a tissue- and time-restricted manner, for example by expressing a wild-type version of a gene and inducing the expression of the mutated version, or vice-versa. This innovative strategy is based on the reduction of the homology between the sequences in opposite orientations while maintaining almost identical the amino acid sequence of the encoded polypeptide.

Thus, in a first aspect, the present invention relates to a conditional knock-in cassette which is a double stranded DNA molecule comprising a sequence A, a sequence B, a first pair RTS1 and RTS1′ and a second pair RTS2 and RTS2′ of recombinase target sites (RTS), wherein

(i) RTS of the first pair and RTS of the second pair are unable to recombine together, and

(ii) RTS1 and RTS1′ are in an opposite orientation, and

(iii) RTS2 and RTS2′ are in an opposite orientation, and

(iv) sequences A and B and RTS are in the following order from 5′ to 3′: RTS1, sequence A, RTS2, sequence B, RTS1′ and RTS2′, and

(v) sequences A and B each comprises at least one coding sequence and said coding sequences are on different DNA strands, and

(vi) the amino acid sequence encoded by sequence A has at least 90% sequence identity to the amino acid sequence encoded by sequence B, and

(vii) the coding strand of sequence A and the non-coding strand of sequence B are unable to hybridize.

Preferably, RTS are recognized by the same recombinase.

RTS may be recognized by a recombinase selected from the group consisting of the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase X Int, the integrase λ Int, the Gin recombinase of the phage Mu, PhiC31 integrase, the Tn3 resolvase, the Dre recombinase, the Tre recombinase, the prokaryotic beta-recombinase, and variants thereof. Preferably, RTS may be recognized by a recombinase selected from the group consisting of the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase X Int, the Gin recombinase of the phage Mu, PhiC31 integrase, and derivatives thereof.

More preferably, RTS are recognized by a recombinase selected from the group consisting of the Cre recombinase of bacteriophage P1 and the FLP recombinase of Saccharomyces cerevisiae, and variants thereof.

Even more preferably, RTS are recognized by the Cre recombinase or a variant thereof.

More preferably, RTS are selected from the group consisting of LoxP site and mutants thereof such as Lox 511, Lox 66, Lox 71, Lox 512, Lox 514, Lox B, Lox L, Lox R, Lox 5171, Lox 2272, m2, m3, m7 and m11.

In some preferred embodiments, RTS1 and RTS1′ are LoxP sites and RTS2 and RTS2′ are Lox 511 sites, or vice-versa.

In the cassette of the invention, said at least one coding sequence of sequence A and/or sequence B may be an exon or a fragment thereof.

Preferably, the amino acid sequence encoded by sequence A has at least 95%, preferably at least 99%, sequence identity to the amino acid sequence encoded by sequence B.

More preferably, the amino acid sequence encoded by sequence A differs from the amino acid sequence encoded by sequence B by only one amino acid.

The coding strand of sequence A may have less than 60%, preferably less than 55%, 50%, 45%, 30% or 20% sequence identity to the coding strand of sequence B.

The coding sequence(s) of sequence A may have less than 70%, preferably less than 60%, 50% or 40%, identity to the coding sequence(s) of sequence B, and the non-coding sequence(s) of sequence A may have less than 30%, preferably less than 20%, 10% or 5%, identity to the non-coding sequence(s) of sequence B.

Preferably, the pre-mRNA obtained from the conditional knock-in cassette has a frequency of the minimum free energy RNA secondary structure of 0 and/or an ensemble free energy higher than −800 kcal/mol.

The cassette of the invention may further comprise an additional coding sequence, preferably encoding a reporter protein or a selection marker.

In another aspect, the present invention also relates to a vector comprising a conditional knock-in cassette of the invention.

In a further aspect, the invention relates to an isolated transgenic host cell, preferably excluding human embryonic cell, comprising a conditional knock-in cassette of the invention. It also relates to a transgenic organism, preferably excepted humans, comprising at least transgenic cell of the invention, preferably a transgenic mouse.

In another aspect, the present invention also relates to a method, preferably an in vitro method, of generating a conditional knock-in allele of a target gene in a cell, the method comprising

introducing into the cell a conditional knock-in cassette or a vector of the invention, and

obtaining a transgenic cell in which the conditional knock-in cassette is inserted by homologous recombination into the genome.

### BRIEF DESCRIPTION OF THE DRAWINGS

FIG. 1: Principle of the FLEx switch system. The top of the picture (before Cre mediated inversion/excision) represents the conditional allele which express the wild type form of a gene. The rearrangement mediated by the Cre recombinase takes place in two steps. The first step consists in the inversion of the sequence between the LoxP or the sequence between Lox511. The second step (which is concomitant with the first step) consists in the excision (suppression) of the fragment between two lox sites positioned in the same direction. Finally, the original exon A is abolished and replaced by the exon B.

FIG. 2: conditional Kif2a FLEx switch. The scheme represents a conditional allele expressing the wild type form of Kif2a (upper panel). Upon Cre-mediated rearrangement the wild type exon is removed and replaced by the exon containing the mutant form of the protein.

FIG. 3: RT-PCR on ES cell clones. Well 1: Wild type ES cells pellet S3_WT (passage11), derived from wild type C57Bl/6N mouse. Well 2: K540353_26 ES cells pellet (passage26)=heterozygous targeted ES cell clone with mutation sequence inversed: 3′←5′ (mutated exon 10 inversed in Kif2a mouse locus). Well 3: K5403S3_26 cre9 ES cells pellet (passage33)=ES cell clone after Cre mediated inversion/excision with mutation in the correct orientation 5′->3′ (mutated exon 10 in Kif2a mouse locus). Well 4: Negative control.

FIG. 4: The mRNA expression of Kif2a WT in ES cells, and KIF2A mutant in ES cells. A: Kif2a WT mRNA expression (%) in model ES cells before the action of the Cre (−Cre), after the action of the Cre (+Cre) and WT ES cells. B: Mutant KIF2A mRNA expression (%) on WT ES cells, model ES cells before the action of the Cre (−Cre) and after the action of the Cre (+Cre).

FIG. 5: Validation of the approach based on KIF2A protein expression in modified ES cells. A and B control immunofluorescence staining of KIF2A (green) and α-tubulin (red) in wild-type and p.His321Asp patient-derived fibroblasts showing the abnormal localization of mutant KIF2A. In A note that instead of the expected diffuse punctiform cytoplasmic and nuclear distribution (as observed for wild-type KIF2A), KIF2A mutants showed a predominant colocalization with and decoration of microtubules. B: Immunofluorescent images of metaphasic fibroblasts expressing wild type or mutant. KIF2A and stained against KIF2A (red), β-tubulin (blue) and β-tubulin (green). Note that mutant KIF2A localisation is altered in mitotic spindle of the patient's fibroblasts. C: Immunofluorescence staining of KIF2A in modified ES cells (including in mitosis) before the action of the Cre (−Cre) and after the action of the Cre (+Cre). Note that the abnormal localization of KIF2A at the spindle poles of ES cells expressing Cre.

FIG. 6: Expression of WT Kif2a in the brain of the mouse model with the inverted sequence (in the absence of Cre-recombinase) and after removal of the selection cassette (frt-neo cassette) (FIK2A bar) in comparison to the expression in control brain (WT bar).

### DETAILED DESCRIPTION OF THE INVENTION

The FLEx switch system was extensively described in the article of Schnütgen et al., (supra) as well as in the international patent application WO 02/088353.

Briefly, this system relies on the property of Cre recombinase to both invert and excise any intervening DNA flanked by two loxP sites placed in opposite and identical orientations, respectively, and on the use of loxP mutant sites that can recombine with themselves but not with wild type loxP sites.

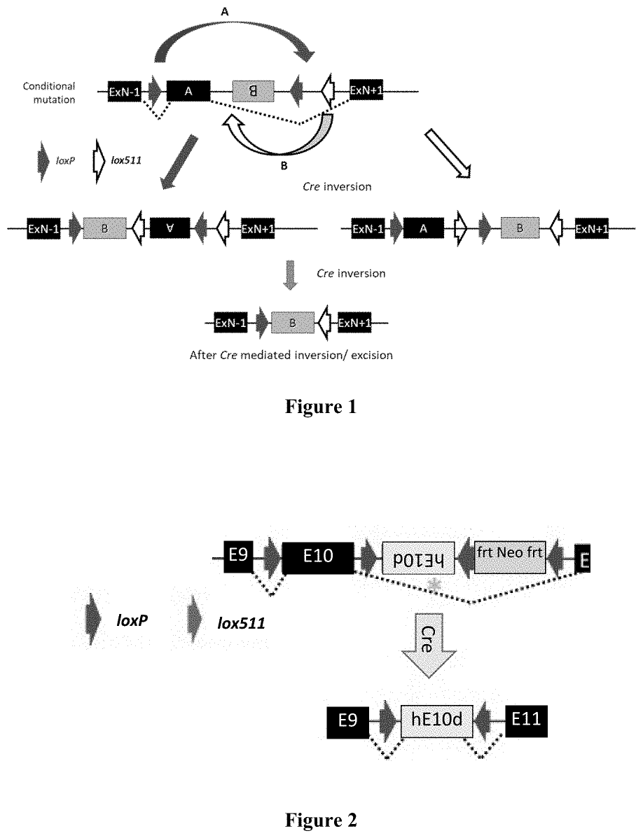

The principle of this system is illustrated in FIG. 1. The rearrangement mediated by the Cre recombinase takes place in two steps. The first step consists in the inversion of the sequence between the LoxP sites or the sequence between Lox511 sites. The second step (which is concomitant with the first step) consists in the excision (deletion) of the DNA fragment between the two lox sites in the same orientation. Finally, the expression of a coding sequence is turned off while the expression of another one is concomitantly turned on.

As illustrated in the experimental section, the inventors showed that this system was unable to generate conditional point mutation models if the point mutation is not located in the last exon. Indeed, they revealed that transcripts expressed by the engineered allele lack the normal exon even in the absence of Cre-recombinase expression thereby mimicking constitutive knock-out. Without being bound by this theory, they assumed that the pre-mRNA obtained from the engineered allele contains a secondary structure that may lead to a splicing event encompassing the normal exon.

The inventors herein found that the FLEx switch system can be used to generate conditional point mutation models by reducing the homology between the sequences in opposite orientations while maintaining almost identical the amino acid sequence of the encoded polypeptide. This approach offers the possibility of creating a conditional knock-in model with the desired mutation at any position in the gene and at any time. This strategy offers also the possibility to develop point mutation models and to assess phenotype reversibility in a tissue- and time-restricted manner, for example by expressing a wild-type version of a gene and inducing the expression of the mutated version, or vice-versa. This innovative strategy is thus expected to cover a real need in many fields of genetics, biology and biomedical research and could be implemented as a “universal” strategy to generate conditional knock-in models.

According, in a first aspect, the present invention relates to a conditional knock-in cassette. The cassette of the invention is designed to generate, after introduction into a host cell and integration into the genome, preferably by homologous recombination, a conditional knock-in allele of a gene.

The conditional knock-in cassette of the invention is a double stranded DNA molecule comprising a sequence A, a sequence B, a first pair RTS1 and RTS1′ and a second pair RTS2 and RTS2′ of recombinase target sites (RTS), wherein

(i) RTS of the first pair are unable to recombine with RTS of the second pair, and vice-versa,

(ii) RTS1 and RTS1′ are in an opposite orientation, and

(iii) RTS2 and RTS2′ are in an opposite orientation, and

(iv) sequences A and B and RTS are in the following order from 5′ to 3′: RTS1, sequence A, RTS2, sequence B, RTS1′ and RTS2′, and

(v) sequences A and B each comprises at least one coding sequence and said coding sequences are on different DNA strands, and

(vi) the amino acid sequence encoded by sequence A has at least 90% sequence identity to the amino acid sequence encoded by sequence B, and

(vii) the coding strand of sequence A and the non-coding strand of sequence B are unable to hybridize.

In said cassette, coding sequence(s) of sequence A encode the amino acid sequence expresses in the host cell before recombinase induction, preferably the amino acid sequence expresses in the host cell before introduction of said cassette into its genome, and sequence B corresponds to the sequence that will be expressed in place of sequence A after induction.

As used herein, the term “DNA molecule” means a single- or double-stranded deoxyribonucleic acid, preferably double-stranded deoxyribonucleic acid. The deoxyribonucleotides are typically joined by phosphodiester bonds, although in some cases, nucleic acid analogs may also be included and provide alternate backbones.

It should be recognized that the cassette of the invention is not a naturally occurring nucleic acid. However, this cassette may also be referred as an isolated DNA molecule. The term “isolated DNA molecule” refers to a DNA molecule isolated from a source cell and that has been separated from at least about 50 percent of polypeptides, peptides, lipids, carbohydrates, polynucleotides or other materials with which the DNA molecule is found in said source cell. Preferably, an isolated nucleic acid molecule is substantially free from any other contaminating nucleic acid molecules or other molecules that would interfere with its use such as cellular materials or culture medium when produced by recombinant techniques, or chemical precursors or other chemicals when chemically synthesized.

The cassette of the invention comprises at least two pairs of recombinase target sites (RTS), i.e. a first pair RTS1 and RTS1′ and a second pair RTS2 and RTS2′.

As used herein, the term “recombinase target site” refers to a short nucleic acid sequence which serves as site for both recognition and recombination by a site-specific recombinase enzyme. A recombinase target site generally comprises short inverted repeat elements (usually from 11 to 13 bp in length) that flank a spacer region sequence (usually from 6 to 8 bp in length).

Examples of RTS include, but are not limited to, the loxP site and variants thereof recognized by the Cre recombinase of bacteriophage P1, the FRT site and variants thereof recognized by the FLP recombinase of Saccharomyces cerevisiae, attP-, attB-, attL- or attR-sites recognized by the phage integrase ΦC31 or lambda integrase, six-site recognized by the prokaryotic beta-recombinase, gix-site recognized by the Gin recombinase of the phage Mu, the rox site recognized by the Dre recombinase, R-site recognized by the R recombinase of Zygosaccharomyces rouxii and Res-site recognized by the Tn3 resolvase. Preferably, RTS are recognized by a recombinase selected from the group consisting of the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase X Int, the Gin recombinase of the phage Mu, PhiC31 integrase, and variants thereof. More preferably, RTS are recognized by a recombinase selected from the group consisting of the Cre recombinase of bacteriophage P1 and the FLP recombinase of Saccharomyces cerevisiae, and variants thereof.

Recombinase target sites between which a recombinase can catalyse an excision or inversion event are termed matching or compatible recombinase target sites. For example, two LoxP sites constitute a matching pair of RTS and are thus able to recombine together. Inversely, LoxP site and Lox511 are incompatible and are unable to recombine together. As used herein, the term “a pair of RTS” refers to a matching pair of RTS, i.e. two RTS that are recognized by the same recombinase and are able to recombine together.

In the cassette of the invention, RTS of the first pair and RTS of the second pair are unable to recombine together. As used herein, the term “unable to recombine” does not necessarily mean that absolutely no recombination event can occur. This term indicates that RTS of two different pairs, i.e. incompatible RTS, do not significantly recombine together or have a markedly reduced rate of recombination together by comparison to the recombination rate with the RTS of the same pair. Preferably, RTS of the first pair and RTS of the second pair do not significantly recombine together.

RTS of the same pair may be identical or different. For example, a pair may consist of Lox 66 and Lox71.

In preferred embodiments, RTS of the same pair are identical, for example two LoxP sites or two Lox511 sites.

As used herein, the terms “recombinase” and “site-specific recombinase” are used interchangeably and refer to an enzyme that recognizes and binds to specific recombinase target sites and catalyzes the recombination of nucleic acids in relation to these sites. These enzymes have both endonuclease and ligase activities and catalyse (i) the deletion of a DNA fragment flanked by compatible RTS in the same orientation (i.e. head-to-head or tail-to-tail), and/or (ii) the inversion of a DNA fragment flanked by compatible RTS in opposite orientation (i.e. head-to-tail or tail-to-head). Preferably, as used herein, the term “recombinase” refers to a recombinase catalysing the deletion of a DNA fragment flanked by compatible RTS in the same orientation and the inversion of a DNA fragment flanked by compatible RTS in opposite orientation.

Examples of recombinases include, but are not limited to, the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1 or Kluyveromyces waltii pKW1, the integrase X Int, the integrase λ Int, the Gin recombinase of the phage Mu, PhiC31 integrase, the Tn3 resolvase, the Tre recombinase, the Dre recombinase (Anastassiadis et al. Disease Models & Mechanisms 2009 2: 508-515), the prokaryotic beta-recombinase, and variants thereof. Preferably, the recombinase is selected from the group consisting of the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase X Int, the Gin recombinase of the phage Mu and PhiC31 integrase, and variants thereof. More preferably, the recombinase is selected from the group consisting of the Cre recombinase of bacteriophage P1 and the FLP recombinase of Saccharomyces cerevisiae, and variants thereof.

Numerous variants of recombinase enzymes have been described in the literature, in particular variants of FLP or Cre recombinase. These variants may be natural or synthetic and may recognize different RTS than the wild-type enzyme (see e.g. Santoro and Schultz, Proc Natl Acad Sci USA. 2002 Apr. 2; 99(7):4185-90 relating the Cre recombinase variants) or may exhibit improved characteristics (e.g. thermostable variants of FLP such as FLPe (Buchholz et al., 1998, Nat Biotechnol. 16, 657-662) or FLPo (Raymond and Soriano, 2007, PLoS ONE 2, e162), Cre recombinases variants with improved accuracy (see e.g. WO 2014/158593), Cre recombinase variants with improved expression in mammal cells (see e.g. U.S. Pat. No. 6,734,295), or tamoxifen-inducible Cre recombinase variants so-called CreER recombinases (e.g. Feil et al., Methods Mol Biol. 2009; 530:343-63).

The two pairs of RTS present in the cassette of the invention may be recognized by different recombinases or by the same recombinase. Preferably, the two pairs of RTS present in the cassette of the invention are recognized by the same recombinase.

In embodiments in which the two pairs of RTS are recognized by different recombinases, the recombinase recognizing and catalysing recombination between RTS1 and RTS1′ is unable to recognize and catalyse recombination between RTS2 and RTS2′, and vice versa. In such case, the cassette has to be contacted, preferably simultaneously, with each recombinase specific of each pair of RTS in order to carry out inversion and deletion steps of the Flex switch system.

In preferred embodiments, the two pairs of RTS are recognized by the same recombinase, i.e. the same recombinase recognizes RTS1, RTS1′, RTS2 and RTS2′ and catalyzes recombination (inversion and deletion) between RTS1 and RTS1′ and between RTS2 and RTS2′.

In a particular embodiment, the cassette comprises at least one pair of RTS recognized by the Cre recombinase or a variant thereof. Preferably, RTS1/RTS1′ and RTS2/RTS2′ are recognized by the Cre recombinase or a variant thereof.

Cre recombinase and variants thereof recognize loxP site or mutants thereof. LoxP site consists of a sequence comprising an asymmetric 8 bp sequence (or spacer region) between two 13 bp palindromic arms (recognition regions), i.e. 5′-ATAACTTCGTATAATGTATGCTATACGAAGTTAT-3′ (SEQ ID NO: 1). Numerous mutant LoxP sites have been described (see e.g. for review Missirlis et al. BMC Genomics 2006, 7:73). Indeed, differences in palindromic or spacer regions of lox sites, either naturally occurring or randomly mutated can confer specificity to Cre recognition. Example of mutant LoxP sites include, but are not limited to, Lox 511 (ATAACTTCGTATAATGTATACTATACGAAGTTAT; SEQ ID NO: 2), Lox 66 (ATAACTTCGTATAATGTATGCTATACGAACGGTA; SEQ ID NO: 3), Lox 71 (TACCGTTCGTATAATGTATGCTATACGAAGTTAT; SEQ ID NO: 4), Lox 512, Lox 514, Lox B, Lox L, Lox R, Lox 5171 (ATAACTTCGTATAATGTGTACTATACGAAGTTAT; SEQ ID NO: 5), Lox 2272 (ATAACTTCGTATAAAGTATCCTATACGAAGTTAT; SEQ ID NO: 6), m2 (ATAACTTCGTATAAGAAACCATATACGAAGTTAT; SEQ ID NO: 7), m3 (ATAACTTCGTATATAATACCATATACGAAGTTAT; SEQ ID NO: 8), m7 (ATAACTTCGTATAAGATAGAATATACGAAGTTAT; SEQ ID NO: 9) and m11 (ATAACTTCGTATACGATACCATATACGAAGTTAT; SEQ ID NO: 10).

Spacer mutants such as Lox 511, lox 5171, lox 2272, m2, m3, m7 and m11 recombine readily with themselves but have a markedly reduced rate or do not recombine with the wild type site. Such mutants are particularly useful in the present invention. In particular, the first pair of RTS may be a wild-type loxP site while the second pair is a spacer mutant as defined above, or vice-versa. In a particular embodiment, RTS1 and RTS1′ are loxP sites and RTS2 and RTS2′ are lox511 sites, or vice-versa.

In another particular embodiment, the cassette comprises at least one pair of RTS recognized by the Flp recombinase or a variant thereof. Preferably, RTS1/RTS1′ and RTS2/RTS2′ are recognized by the Flp recombinase or a variant thereof.

Flp recombinase and variants thereof recognize FRT site or mutants thereof. As LoxP site, FRT site consists of a sequence comprising an asymmetric 8 bp sequence (or spacer region) between two 13 bp palindromic arms (recognition regions), i.e. 5′-GAAGTTCCTATAC TTTCTAGA GAATAGGAACTTC-3′ (SEQ ID NO: 11). Numerous mutant FRT sites have been described such as FRT G (GAAGTTCCTATAC TCTCTGGA GAATAGGAACTTC; SEQ ID NO: 12), FRT H (GAAGTTCCTATAC TATCTTGA GAATAGGAACTTC; SEQ ID NO: 13; Nakano et al., Nucleic Acids Res. 2001, 29, E40) and FRT F3 sites (GAAGTTCCTATAC TATTTGGA GAATAGGAACTTC; SEQ ID NO: 14; Schlake and Bode, 1994, Biochemistry 33, 12746-12751) that contain double and quadruple mutations in the spacer region and have been reported to show a high recombination efficiency with strict fidelity.

In the cassette of the present invention, RTS1 and RTS1′ are in an opposite orientation and RTS2 and RT2′ are in an opposite orientation. In most of cases, RTS comprise two palindromic recognition regions and the orientation of a RTS sequence is determined by the orientation of its spacer region.

The orientation of RTS drives the activity of the site-specific recombinase. When two RTS of the same pair are in an opposite orientation, the recombinase enzyme catalyzes the inversion of the intervening sequence. This inversion may involve RTS1/RTS1′ or RTS2/RTS2′. After inversion, RTS of one of these pairs (RTS1/RTS1′ or RTS2/RTS2) are in the same orientation, and the recombinase enzyme catalyzes the excision of the intervening sequence.

Sequences A and B each comprises at least one coding sequence. Preferably said at least one coding sequence is a gene or a fragment thereof, such as an exon.

In an embodiment, sequences A and B comprise at least one exon or a fragment thereof.

In a particular embodiment, sequences A and B comprise one exon or a fragment thereof.

These sequences may further comprise one or several non coding sequences, in particular one or several introns or fragments thereof. In a particular embodiment, sequences A and B comprise a coding sequence, e.g. an exon, flanked by one or two non coding regions, e.g. intronic sequences. Preferably, these intronic sequences have a length of more than 200 bp. In particular, the intronic sequences may have a length of 200 bp to 300, 400, 500, 600, 700, 800, 900, 1000 bp. More preferably, the intronic sequences have a length of 300 bp to 500 bp.

In the cassette of the invention, sequences A and B are in an opposite orientation. This means that the coding sequence(s) of sequences A and B are not on the same strand of the double stranded DNA molecule. Preferably, if sequence A or B comprises several coding sequences, all these sequences are in the same orientation.

The present invention relates to the technical problem of generating conditional knock-in alleles. As used herein, the term “knock-in allele” refers to a genetic modification resulting from the replacement of the genetic information encoded in a chromosomal locus with a mutated DNA sequence. The term has to be distinguished from the term “knock-out” referring to a genetic modification resulting from the disruption of the genetic information encoded in a chromosomal locus.

As mentioned above, in the cassette of the invention, sequence A encodes the original amino acid sequence, i.e. to the sequence expresses in the host cell before introduction of said cassette into its genome, and sequence B encodes to the mutated sequence. In particular, the original amino acid sequence may be a wild-type sequence to be mutated or may be a sequence comprising a mutation to be reversed.

In the context of the present invention, the amino acid sequences encoded by sequence A and sequence B have a high degree of identity.

Preferably, the amino acid sequence encoded by sequence A has at least 90% sequence identity to the amino acid sequence encoded by sequence B.

As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/). Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

In particular, the amino acid sequence encoded by sequence A may have at least 91%, 92%, 93%, 94%, 95%, 96%, 97%, 98% or 99% sequence identity to the amino acid sequence encoded by sequence B.

Preferably, the amino acid sequence encoded by sequence A differs from the amino acid sequence encoded by sequence B by less than 20 amino acid residue(s).

More preferably, the amino acid sequence encoded by sequence A differs from the amino acid sequence encoded by sequence B by 1, 2, 3, 4, 5, 6, 7, 8, 9, 10, 11, 12, 13, 14 or 15 amino acid residue(s).

In a particular embodiment, the amino acid sequence encoded by sequence A differs from the amino acid sequence encoded by sequence B by less than 5 amino acid residues, preferably by only one amino acid residue.

Amino acid difference(s) may be due to substitution, insertion, or deletion, or combinations thereof.

As specified above, the amino acid sequences encoded by sequence A, i.e. the original sequence, and sequence B, i.e. the mutated one, have a high degree of identity. Usually, in order to introduce some mutations in an amino acid sequence, the person skilled in the art mutates only few nucleotides corresponding to the codons of interest in the coding nucleotide sequence. However, in the present application, the inventors demonstrated that applying this routine technique resulted in a constitutive knock-out allele and not in a conditional knock-in allele.

The inventors found that while the amino acid sequences encoded by sequence A and sequence B may show a high degree of identity, the coding strand of sequence A has to be unable to hybridize with the non-coding strand of sequence B, thereby preventing the formation of secondary structure such as hairpin structure. As sequences A and B are in an opposite orientation, this also means that sequence A and sequence B of the same strand cannot form a hairpin together, i.e. that the pre-mRNA cannot form a hairpin structure.

Reducing the identity between sequences A and B may be obtained by acting on coding and/or non-coding sequences of sequence A and/or sequence B.

Nucleotide sequence variations may be introduced into coding sequence(s) of sequence A and/or sequence B using synonymous (or silent) mutations. Indeed, exploiting the redundancy of the genetic code (some amino acids are coded for by 2, 3, 4, or 6 different codons), it is possible to introduce changes in the nucleotide sequence without impacting the amino acid sequence.

Such variations may be easily obtained by replacing a coding sequence with the corresponding orthologous gene or gene fragment, e.g. exon, found in another species. Preferably, this orthologous gene or gene fragment is further degenerated in order to prevent hybridization between the coding strand of sequence A and the non-coding strand of sequence B, i.e. to prevent the formation of an hairpin in the pre-mRNA. As used herein, the term “degenerated” means introducing synonymous mutations using the redundancy of the genetic code. For example, and as illustrated in the experimental section, if the coding sequence of sequence A is exon 10 of the mouse Kif2a gene, the coding sequence of sequence B may be exon 10 of the human Kif2a gene which has been further degenerated and shows only 42% identity with exon 10 of the mouse Kif2a gene.

Alternatively, or preferably in addition, nucleotide sequence variations may be introduced into non-coding sequence(s) of sequence A and/or sequence B.

In preferred embodiments, non-coding sequences found in sequences A and B are intronic sequences. Variations in such non coding sequences may be obtained for example by random or targeted mutagenesis, or by replacing the intronic sequence with an intron from another locus, with another intron of the same locus, or with an intron of another species, e.g. the intronic sequence of the corresponding intron found in the orthologous gene of another species.

For example, if sequence A comprises exon 10 of the mouse Kif2a gene flanked by two intronic sequences, sequence B may comprise degenerated exon 10 of the human Kif2a gene as described above flanked by two human intronic sequences. If necessary, such intronic sequences may be further mutated.

Preferably, variations in non-coding sequences preserve splicing signals such as the splice donor site (5′ end of the intron), the splice branch site (near the 3′ end of the intron) and the splice acceptor site (3′ end of the intron) which are required for correct splicing of the pre-mRNA. Numerous bioinformatics tool are known by the skilled person and may be used to predict splicing signals and splicing events such as GeneSplicer (http://ccb.jhu.edu/software/genesplicer/) or Spliceport (http://spliceport.cbcb.umd.edu/).

Preferably, the coding strand of sequence A is unable to hybridize with the non-coding strand of sequence B, even in conditions of low stringency, in order to prevent hairpin formation at the pre-mRNA level. More preferably, the coding strand of sequence A has less than 60%, less than 55%, 50%, 45%, 30% or less than 20% sequence identity to the coding strand of sequence B. More particularly, the coding sequence(s) of sequence A has (have) less than 70%, preferably less than 60%, 50% or 40%, identity to the coding sequence(s) of sequence B, and the non-coding sequence(s) of sequence A has (have) less than 30%, preferably less than 20%, 10% or 5%, identity to the non-coding sequence(s) of sequence B.

Alternatively, the incapacity of the coding strand of sequence A to hybridize with the non-coding strand of sequence B can be assessed by checking that the pre-mRNA obtained from the cassette of the invention does not form an hairpin, i.e. that sequence A and sequence B on the same strand cannot hybridize and thus cannot form an hairpin. Preferably, the pre-mRNA obtained from the cassette of the invention has a frequency of the minimum free energy RNA secondary structure of 0 and/or an ensemble free energy higher than −800 kcal/mol. More preferably, the pre-mRNA has a frequency of the minimum free energy structure of 0 and an ensemble free energy higher than −800 kcal/mol. The term “minimum free energy RNA secondary structure” (MFE) as used herein means the structure found by thermodynamic optimization (i.e. an implementation of the Zuker algorithm (M. Zuker and P. Stiegler., Nucleic Acids Research 9: 133-148 (1981)) that has the lowest free energy value. The term “frequency of the minimum free energy RNA secondary structure” refers to the fraction of the MFE structure in the thermodynamic ensemble: (eA(−E/kT))/Z, where E is the minimum free energy of the structure, k is the Boltzmann constant, T is the temperature and Z is the partition function (Wuchty et al, Biopolymers 49: 145-165 (1999)). The term “ensemble free energy” as used herein means (−kT ln(Z)) in kcal/mol where k, T, and Z are defined as above and implemented e.g. in the ViennaRNA software package (I. L. Hofacker et al, Monatsh. Chem., 125: 167-188 (1994)). The ensemble free energy is defined by J. S. McCaskill in Biopolymers 29: 1105-11 19 (1990). The frequency of the MFE structure as well as the ensemble free energy can be easily calculated by the skilled person using any software implementing the Zuker algorithm such as the program RNAfold (http://www.tbi.univie.ac.at/RNA/).

In a preferred embodiment, sequence A corresponds to the original sequence, i.e. to the nucleotide sequence found in the host cell before introduction of the cassette of the invention into its genome. In this embodiment, nucleotide variations in order to prevent hairpin formation at the pre-mRNA level are only carried out on sequence B. This means that coding sequence(s) of sequence B is(are) degenerated and/or non coding sequence(s) of sequence B is(are) mutated and/or replaced or vice-versa.

In particular, sequence A may comprise the original sequence comprising an exon flanked by two intronic sequences, and sequence B may comprise a “degenerated” exon comprising the mutation(s) of interest flanked by two mutated or replaced intronic sequences, e.g. two intronic sequences of the corresponding introns found in the orthologous gene of another species.

In the cassette of the present invention, sequences A and B and RTS are in the following order from 5′ to 3′: RTS1, sequence A, RTS2, sequence B, RTS1′ and RTS2′.

These elements may be immediately adjacent from each other or separated by a nucleotide sequence, e.g. a spacer region. In particular, these spacers may comprise restriction sites.

In some embodiments, the cassette of the invention may comprise an additional coding sequence, preferably between RTS1′ and RTS2′.

Preferably, this coding sequence is suitable for selecting host cells comprising a DNA molecule of the invention. In particular, this coding sequence may encode a reporter protein or a selection marker. By “reporter protein” as used herein is meant a protein that provides a detectable signal, either directly or indirectly, e.g. after reaction with a substrate. Examples of reporter proteins include, but are not limited to, fluorescent proteins such as green fluorescence protein (GFP) and variants thereof, β-galactosidase, β-glucuronidase, alcaline phosphatase, luciferase, alcohol dehydrogenase and peroxidase.

Preferably, this sequence codes for a selection marker which is useful to select rare homologous recombination events in ES cells. By “selection marker” as used herein is meant a marker allowing selection of a host cell comprising the DNA molecule of the invention and expressing said marker. Examples of genes encoding selection markers include, but are not limited to, antibiotic resistance genes such as neomycine, puromycine or hygromycine resistance gene.

Optionally, said additional coding sequence may be flanked by two compatible RTS in the same orientation. These two additional RTS should not interfere, i.e. are not compatible, with RTS1/RTS1′ and RTS2/RTS2′ and are preferably recognized by a different recombinase. For example, RTS1/RTS1′ and RTS2/RTS2′ may be recognized by Cre recombinase whereas RTS flanking the additional coding sequence may be recognized by FLP recombinase.

The cassette of the present invention is a conditional knock-in cassette. This means that, after introduction of said cassette into the genome of a host cell, the original allele still expresses the original form of the gene of interest.

Preferably, before recombinase-mediated rearrangement, splicing of the primary transcript obtained from the locus comprising the cassette of the invention, eliminates RTS1, RTS2, sequence B, RTS1′ and RTS2′. Splicing signals allowing such elimination are preferably encompass/preserve in the cassette of the invention, in particular a splice acceptor site at the 5′ end of the coding strand of sequence A and/or a splice donor site at the 3′ end of the coding strand of sequence A.

Similarly, after recombinase-mediated rearrangement, the correct splicing of the primary transcript may involve splicing signals encompass in the cassette of the invention, in particular a splice acceptor site at the 5′ end of the coding strand of sequence B and/or a splice donor site at the 3′ end of the coding strand of sequence B.

Splicing events as well as splicing signals to be introduced in the cassette of the invention may be easily defined by the skilled person, in particular using bioinformatics tools such as such as GeneSplicer (http://ccb.jhu.edu/software/genesplicer/) or Spliceport (http://spliceport.cbcb.umd.edu/).

In a second aspect, the present invention also provides a vector comprising a conditional knock-in cassette of the invention and as described above.

All embodiments described above for the cassette of the invention are also contemplated in this aspect.

By “vector” is meant a nucleic acid molecule, preferably a DNA molecule derived, for example, from a plasmid, bacteriophage or virus, into which a nucleic acid sequence may be inserted or cloned. Non-limiting examples of vectors include plasmids, phages, cosmids, phagemids, yeast artificial chromosomes (YAC), bacterial artificial chromosomes (BAC), human artificial chromosomes (HAC), viral vectors such as adenoviral vectors or retroviral vectors, and other DNA sequences which are conventionally used in genetic engineering and/or able to convey a desired DNA sequence to a desired location within a host cell.

A vector preferably contains one or more restriction sites and may be capable of autonomous replication in a defined host cell including a target cell or tissue or a progenitor cell or tissue thereof, or be partially or entirely integrable with the genome of the defined host such that the cloned sequence is reproducible. Accordingly, the vector may be an autonomously replicating vector, i.e. a vector that exists as an extrachromosomal entity, the replication of which is independent of chromosomal replication, e.g. a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The choice of the vector will typically depend on the compatibility of the vector with the host cell into which the vector is to be introduced.

The vector may also include a selection marker such as an antibiotic resistance gene that can be used for selection of suitable transformants, primer sites (e.g. for DNA amplification or sequencing) as well as one or several control sequences. The term “control sequences” means nucleic acid sequences necessary for expression of a gene. Such control sequences include, but are not limited to, promoters, IRES (internal ribosome entry sites), transcriptional or translational initiation sites, and transcription terminator.

In a particular embodiment, the vector of the invention is a targeting vector, i.e. a vector that comprises the nucleic acid sequences that are to be integrated into the genome of the cell as well as the elements that are required to enable site-specific recombination.

In particular, the targeting vector may comprise a cassette of the invention flanked by two arms of homology allowing site specific integration of the cassette of the invention into the genome of a host cell. These homology arms correspond to the regions flanking the sequence A in the genome of the host cell. These sequences may be easily chosen by the skilled person depending on the sequence A to be mutated. Homology arms may be more than 100 bp in length, in particular more than 100, 200, 500, 1000, 1500, 2000, 2500, 3000, 3500 or 4000 bp in length. Preferably, homology arms are about 2500 bp in length. These homology arms are preferably more than 95%, more than 99 or 100% homologous to the wild-type sequences flanking sequence A in the genome of the host cell.

The vector can be synthesized by standard methods. Parts of said vector can be isolated from natural sources and ligated with the remaining parts of the vector using techniques known in the art. Vector modification techniques are described for example in Sambrook and Russel “Molecular Cloning, A Laboratory Manual”, Cold Spring Harbor Laboratory, N.Y. (2001). Furthermore, the cassette of the invention may be cloned in a huge variety of vectors commercially available.

The introduction of the vector into a host cell may be achieved using any of the methods known in the art for introducing nucleic acid molecules into cells. Such methods include for example calcium phosphate-DNA co-precipitation, DEAE-dextran-mediated transfection, electroporation, microinjection, liposome fusion, lipofection, protoplast fusion, retroviral infection, and biolistics. The same methods may be employed for introducing the nucleic acid molecule encoding the recombinase(s) into the cell.

In another aspect, the present invention also relates to the use of the cassette or vector of the invention as a transgene, i.e. the use of the cassette or vector of the invention to transform, transduce or transfect a host cell.

The present invention further relates to an isolated transgenic host cell comprising a cassette or vector of the invention.

All embodiments described above for the cassette and the vector of the invention are also contemplated in this aspect.

Any cell type capable of homologous recombination may be used to practice this invention.

The host cell may be a prokaryotic or eukaryotic cell. Preferably the host cell is a eukaryotic cell, e.g. a yeast or an isolated cell of an animal or plant.

Preferably, the host cell is an isolated cell of an animal, from non-human animals, such as domesticated animals (e.g., cows, sheep, cats, dogs, and horses), primates (e.g., non-human primates such as monkeys), rabbits, fish, rodents (e.g., mice, rats, hamsters, guinea pigs), and non-vertebrates such as flies and worms (e.g., Drosophila melanogaster and Caenorhabditis elegans), or from human. More preferably, the host cell is a mammal cell, even more preferably a murine cell.

The host cell may be a totipotent, pluripotent, or adult stem cell, a zygote, or a somatic cell.

In an embodiment, the host cell is a prokaryotic or eukaryotic cell excluding human embryonic cell. In another embodiment, the host cell is a non-human cell.

The cassette or vector of the invention may be introduced into the host cell by any method known by the skilled person, e.g. any method such as described above.

In preferred embodiments, the cassette of the invention is integrated into the genome of the host cell via homologous recombination thereby providing a conditional knock-in allele of the gene encompassing sequence A, i.e. an allele comprising a cassette of the invention but producing a phenotype that is indistinguishable from that produced by the cognate wild type allele. Any method allowing targeted insertion of a cassette into the genome of the cell may be used by the skilled person.

The methods, cassettes and vectors described herein can be used to create a conditional knock-in allele at any genomic locus. Several cassettes or vectors may also be introduced into the host cell to create conditional knock-in alleles at several genomic loci.

Optionally, the host cell may also comprise a gene encoding a recombinase recognizing RTS1/RTS1′ and/or RTS2/RTS2′, preferably recognizing RTS1/RTS1′ and RTS2/RTS2′, under the control of an inducible promoter. Preferably said inducible promoter is a tissue-specific promoter.

The present invention further relates to a method, preferably an in vitro method, of generating a conditional knock-in allele in a cell comprising a target gene, the method comprising

introducing into the cell a conditional knock-in cassette or vector of the invention, and

obtaining a transgenic cell in which the conditional knock-in cassette has been inserted by homologous recombination into the genome.

The target gene is the gene encompassing sequence A as defined above.

Selection of transgenic cells comprising the cassette or vector of the invention may be performed by any method known by the skilled person, for example using a reporter protein or selection marker expressed from the cassette or the vector.

The present invention also relates to a method of generating a knock-in allele in a cell comprising a target gene, the method comprising

introducing into the cell a conditional knock-in cassette or vector of the invention, and

obtaining a transgenic cell in which the conditional knock-in cassette has been inserted by homologous recombination into the genome, and

contacting said conditional knock-in cassette with one or several recombinase(s) recognizing RTS1/RTS1′ and RTS2/RTS2′, thereby inducing the excision of sequence A and its replacement by sequence B.

The target gene is the gene encompassing sequence A as defined above.

Homologous recombination may be performed with or without the help of nucleases routinely used for such recombination such as ZFNs, TALE nucleases, CRISPR/Cas9.

The step of contacting the conditional knock-in cassette with the recombinase(s) may be performed via several methods:

i) when the host cell comprises a gene encoding the recombinase(s) under the control of an inducible promoter, the expression of the recombinase(s) may be induced by various methods depending on the nature of the inducible promoter. For example, this expression may be induced by adding doxycycline, tetracycline, RU486 and/or tamoxifen to the culture medium; and/or

ii) a nucleic acid encoding the recombinase(s) may be introduced into the host cell. Preferably, the nucleic acid encoding the recombinase(s) is contained in an expression vector, i.e. is placed in an expression vector under to control of a promoter. The expression vector may be maintained in the cell in an episomal form or may be stably integrated into the genome; and/or

iii) the recombinase(s) may be directly introduced into the host cell, e.g. by liposome fusion.

The present invention further relates to a transgenic organism, preferably a non-human transgenic organism comprising at least one transgenic host cell of the invention. The invention also relates to a method of generating a transgenic organism comprising at least one transgenic host cell of the invention.

All embodiments described above for the cassette, vector and transgenic cell of the invention are also contemplated in this aspect.

In particular, the organism may be a non-human animal, such as domesticated animals (e.g., cows, sheep, cats, dogs, and horses), primates (e.g., non-human primates such as monkeys), rabbits, fish, rodents (e.g., mice, rats, hamsters, guinea pigs), and non-vertebrates such as flies and worms (e.g., Drosophila melanogaster and Caenorhabditis elegans). Preferably, the transgenic organism is a non-human mammal. More preferably, the transgenic organism is a mouse.

Methods of generating transgenic organisms, in particular transgenic mice are well-known by the skilled person. It should be understood that any of these methods can be used to practice the invention and that the methods disclosed herein are non-limitative.

In particular, the method of generating a transgenic organism may comprise

introducing a cassette or vector of the invention in an embryonic stem cell, preferably a non-human embryonic stem cell,

obtaining a transgenic embryonic stem cell wherein the cassette of the invention is inserted into the genome by homologous recombination,

injecting said transgenic embryonic stem cell into a blastocyst of an animal, preferably a non-human animal, to form chimeras, and

reimplanting said injected blastocyst into a foster mother.

Homologous recombination may be performed with or without the help of nucleases routinely used for such recombination such as ZFNs, TALE nucleases, CRISPR/Cas9. This nuclease can be introduced with the cassette or vector of the invention in the embryonic stem cell.

Embryonic stem (ES) cell are typically obtained from pre-implantation embryos cultured in vitro. Preferably, the cassette or vector of the invention is transfected into said ES cell by electroporation. The ES cells are cultured and prepared for transfection using methods known in the related art. The ES cells that will be transfected with the cassette or vector of the invention are derived from embryo or blastocyst of the same species as the developing embryo or blastocyst into which they are to be introduced. ES cells are typically selected for their ability to integrate into the inner cell mass and contribute to the germ line of an individual when introduced into the animal in an embryo at the blastocyst stage of development. In one embodiment, the ES cells are isolated from the mouse blastocysts.

After transfection into the ES cells, the cassette of the invention integrates with the genomic DNA of the cell in order to create a conditional knock-in allele of a target gene. Preferably, the insertion occurs by homologous recombination wherein homology arms of the vector hybridize to the homologous sequences in the ES cell and recombine to incorporate the cassette of the invention into the endogenous gene encompassing sequence A.

After transfection, the ES cells are cultured under suitable condition to detect transfected cells. For example, when the cassette comprises a marker gene, e.g. an antibiotic resistant marker, e.g. neomycin resistant gene, the cells are cultured in that antibiotic. The DNA and/or protein expression of the surviving ES cells may be analyzed using Southern Blot technology in order to verify the proper integration of the cassette.

In a particular embodiment, the marker gene, e.g. the antibiotic resistant marker, may be then removed, i.e. by contacting the cassette with a recombinase recognizing RTS flanking said marker.

The selected ES cells are then injected into a blastocyst of an animal, preferably a non-human animal, to form chimeras. The non-human animal is preferably a mouse, a hamster, a rat or a rabbit. More preferably, the non-human animal is a mouse.

In particular, the ES cells may be inserted into an early embryo using microinjection. For microinjection, 10 to 20 ES cells are collected into a micropipette and injected into 3 to 5 day old blastocysts recovered from female mice. The injected blastocysts are re-implanted into a foster mother. When the progenies are born, they are screened for the presence of the cassette of the invention, e.g. using Southern Blot and/or PCR technique. The heterozygotes are identified and are then crossed with each other to generate homologous knock-in animals.

In a preferred embodiment, knock-in animals, i.e. animals comprising a cassette of the invention, are crossed with animals comprising the gene(s) of the recombinase(s) recognizing RTS1/RTS1′ and RTS2/RTS2′ placed under the control of a promoter, preferably an inducible promoter. Progenies are then screened to select animals comprising (i) the cassette of the invention and (ii) the gene(s) of the recombinase(s). Preferably, the heterozygotes are identified and are then crossed with each other to generate homologous conditional knock-in animals.

In another embodiment, the ES cells are also transfected with a nucleic acid sequence encoding the recombinase(s) recognizing RTS1/RTS1′ and RTS2/RTS2′, placed under the control of an promoter, preferably an inducible promoter. Preferably, said nucleic acid sequence is also integrated into the genome, preferably by homologous recombination. The promoter may be tissue-specific. Various inducible promoters well-known by the skilled person may be used in the present invention.

In another embodiment, the method of generating a transgenic organism may comprise

introducing in a fertilized egg, preferably a non-human fertilized egg, (i) a cassette or vector of the invention and (ii)

a nuclease system used to target the cassette or vector at the correct locus by homologous recombination,

obtaining a transgenic fertilized egg wherein the cassette of the invention is inserted into the genome by homologous recombination, and

reimplanting said injected fertilized egg into a foster mother.

The nuclease system used to target the cassette or vector at the correct locus may be any suitable system known by the skilled person, such as systems involving ZFN, TALE or CRISPR/Cas9 nucleases.

Preferably, the nuclease system is a CRISPR/Cas9 system. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA or target specific gRNA(s) can be delivered to the cell (these RNAs can alternatively be produced by a gene constructed to express these RNAs). Selection of target sites and designed of crRNA/gRNA are well known in the art.

The present invention also provides cells or tissues, including immortalized cell lines and primary cells or tissues, derived from the transgenic animal, preferably the transgenic non-human animal, of the invention and its progeny.

The present invention further relates to a method of generating a knock-in allele in a transgenic animal, i.e. a knock-in animal model, the method comprising

generating a transgenic organism as described above, i.e. comprising at least one transgenic host cell of the invention, said cell further comprising a nucleic acid sequence encoding the recombinase(s) recognizing RTS1/RTS1′ and RTS2/RTS2′, placed under the control of an inducible or non-inducible promoter, and

when an inducible promoter is used, inducing the expression of the recombinase(s), e.g. by supplementing animal's diet with a substance such as doxycycline, tetracycline, RU486 or tamoxifen, said substance being selected depending on the nature of the inducible promoter.

In some particular embodiments, the promoter is an inducible or non-inducible tissue-specific promoter.

As used herein, the verb “to comprise” is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” refers to a range of values ±10% of the specified value. For example, “about 20” includes ±10% of 20, or from 18 to 22. Preferably, the term “about” refers to a range of values ±5% of the specified value.

All patent and literature references cited in the present specification are hereby incorporated by reference in their entirety.

### Examples

The inventors used previously the Flex switch system illustrated in FIG. 1 on several projects to carry out conditional point mutations. However, in all of these projects involving different genes, they observed the absence of the original sequence A and/or the sequence B (B being the same sequence than A except the desired mutation) in the mRNA. Due to this lack of sequence A (and/or B) in the mRNA, the use of the Flex switch system more often led to a Knock-Out animal instead of a wild type animal.

As illustration, the inventors wanted to generate a model with a conditional point mutation. Sequence A and sequence B (B being the same sequence than A except the desired point mutation) were cloned in forward and reverse orientation into a targeting construct. After electroporation, ES cells were validated (by LR-PCR and Southern blot), chimeras were obtained and germ line transmission was achieved. Heterozygous and homozygous conditional and non-conditional animals were obtained and analyzed by RT-qPCR. The analyze of the total mRNA clearly showed the absence of wild type mRNA in homozygous mice (cKI/cKI) (cKI: conditional knock-in) before Cre mediated inversion/excision whereas the homozygous KI (after Cre mediated inversion/excision) had an expression close to the wild type mouse (WT/WT). When an RT-PCR reaction was performed with a forward primer located in exon N−1 and the reverse primer on exon N+1, an unexpected band appeared when the cKI allele was present. Sequencing of this fragment clearly showed the absence of exon A (transcripts lacking exon A). The same analyzes were performed on different standard FleX models with the same results: unexpected conditional mRNAs were detected leading to the equivalent of a knock-out instead of conditional knock-in.

The inventors developed two conditional knock-in mouse models to study consequences of KIF2A and NEDD4L disease causing mutations associated with malformation of cortical development (MCD). It is worth mentioning that MCD-related to these two genes result exclusively from de novo missense mutations and no loss-of-function mutations was identified. The conditional Kif2a and Nedd41 mouse models correspond to the KIF2A mutation c.961C>G, p.His321Asp detected in a patient with pachygyria and microcephaly (Poirier et al., 2013 Nature Genetics 45:639-647), and NEDD4L mutation c.G2973A; p.R897Q, shown in human to be associated with periventricular nodular heterotopia (PNH) (Broix et al., 2016 Nature Genetics 48:1349-1358).

Design and Generation of the Constructs

In order to develop the conditional knock-in Kif2a mouse model, we generated a plasmid construct (that was subsequently used for electroporation in ES cells) containing one pair of wild-type loxP sites and one pair of lox511 sites, with an alternate organization a head-to head orientation within each pair of sites. The plasmid contained the DNA encoding the mouse exon 10 sequence and mouse intronic flanking sequences in the sense orientation; and the modified “degenerated” human sequence of exon 10 bearing the mutation (c.961C>G, p.His321Asp) and its flanking intronic sequences in the antisense orientation (FIG. 2). Using this strategy, sequence comparison between WT and mutated exons showed that the homology was decreased from 96% homology (for the human sequence) and 100% homology (for the mouse sequence) to only 42% homology. Bioinformatic simulations predicted this “neo-exon”, with significantly reduced homology with the mouse sequence, flanked by human intronic sequences could be spliced at the expected canonical splicing sites.

As illustrated in FIG. 2, initially, the promoter directs the expression of the wild type Kif2a. In this setting, both loxP and lox511 sites are recognized by Cre-recombinase; however lox511 sites recombine efficiently with themselves but not with loxP sites. Thus, Cre-mediated recombination first induces inversion of the DNA at either loxP or lox511 sites generating a repeat of either two loxP or two lox511 sites (see FIG. 1). Further Cre-mediated excision then results in the elimination of the DNA sequence contained between the two loxP or lox511 sites. As a result, the allele construct contains single loxP and lox511 sites making further inversion impossible, and the promoter drives the stable expression of the mutant Kif2a instead of wild type Kif2a.

Results and Validation of the Strategy Using ES Cell Clones and Mouse Models

In order to experimentally check the expression of engineered Kif2a and Nedd41 alleles before and after Cre-recombinase action, we generated ES cell clones and derived cultured cells heterozygous either for the engineered allele (before Cre), or for the mutant allele. We then used these ES clonal cultures to analyze by RT-PCR and quantitative RT-PCR the Kif2a and Nedd41 transcript, and by immunocytochemistry Kif2a protein.

To further check the expression of the engineered alleles, we also used recombinant ES clones to generate chimeric mice and then heterozygous mouse line in which the frt-neo-selection cassette was deleted.

Generation of Conditional Knock-in Kif2a Mice

Knock-in Kif2a mice with the conditional expression of the point mutation were generated in the Institut Clinique de la Souris (Celphedia, Phenomin, ICS, Illkirch) using standard procedures. The Kif2a locus was engineered as follows. A 688 bp wild type genomic fragment comprising exon 10 and surrounding intronic sequences was PCR amplified and subcloned between LoxP and Lox511 sites in an ICS proprietary vector. The basic vector already contains all lox sites in the correct orientation as well as a NeoR cassette surrounded by FRT sites. In a second cloning step, a 529 bps synthetic fragment (String DNA fragment ordered from GeneArt) comprising the degenerated human exon 10 and surrounding human intronic sequences was cloned in an inversed orientation. Both 5′ (4.3 kb) and 3′ (3.2 kb) homology arms were cloned successively. The final construct (FIG. 2) was linearized and electroporated in house derived C57Bl/6N ES cells. Positive clones were selected by Long-Range PCR and further validated by Southern blot using both Neo probe and a 3′ external probe. The fully validated ES cell clone 28 which did not show any abnormalities by ddPCR and karyotype spreading was microinjected in BALB/cN blastocysts, chimeras were obtained and germline transmission of the recombinant allele was achieved in a C57BL/6N pure genetic background. Homozygous Kif2acKI/cKI mice are currently generated by intercrossing Kif2acKI/+ animals.

Generation of Heterozygous Knock-in Kif2a ES by In Vitro Cre Mediated Inversion/Excision

HTN-Cre (6 μM; Excellgen Ref EG-1001) was incubated with fully validated Kif2acKI/+ heterozygous ES cell clone in order to generate the knock-in allele. Inversion/excision of the wild type exon 10 was confirmed by LR-PCR and Sanger sequencing. The resulting ES cells were heterozygous for the KI (introduction of the expected point mutation in the degenerated human exon 10).

For NEDD4L, similar approach was also applied and details concerning the construct used for microinjection and characterization of its bona fide integration in ES cells are herein provided:

Generation of Conditional Knock-in Nedd4l Mice

Knock-in Neddl4 mice with the conditional expression of the point mutation were generated in the Institut Clinique de la Souris (Celphedia, Phenomin, ICS, Illkirch) using standard procedures. The Neddl4 locus was engineered as follows. A 700 bp wild type genomic fragment comprising exon 29 and surrounding intronic sequences was PCR amplified and subcloned between LoxP and Lox511 sites in an ICS proprietary vector. The basic vector already contains all lox sites in the correct orientation as well as a NeoR cassette surrounded by FRT sites. In a second cloning step, a 608 bps synthetic fragment (String DNA fragment ordered from GeneArt) comprising the degenerated human exon 29 and surrounding human intronic sequences was cloned in an inversed orientation. Both 5′ (3.7 kb) and 3′ (3.4 kb) homology arms were cloned successively. The final construct was linearized and electroporated in house derived C57Bl/6N ES cells. Positive clones were selected by Long-Range PCR and further validated by Southern blot using both Neo probe and a 3′ external probe. The fully validated ES cell clone 22 which did not show any abnormalities by ddPCR and karyotype spreading was microinjected in BALB/cN blastocysts, chimeras were obtained and germline transmission of the recombinant allele was achieved in a C57BL/6N pure genetic background. Homozygous Nedd4l cKI/cKI mice were generated by intercrossing Nedd4lcKI/+ animals

Generation of Heterozygous Knock-in Kif2a ES by In Vitro Cre Mediated Inversion/Excision

A plasmid expression Cre was electroporated with fully validated Neddl4 cKI/+ heterozygous ES cell clone (clone 22) in order to generate the knock-in allele. Inversion/excision of the wild type exon 29 was confirmed by LR-PCR and Sanger sequencing. The resulting ES cells were heterozygous for the KI (introduction of the expected point mutation in the degenerated human exon 29).

RT-qPCR on Embryonic Stem (ES) Cells and Mouse Brain

Total RNA was extracted from the different ES cell clones by using TRIzol® reagent (Invitrogen) and the manufacturer's instructions. The purity and the quality of RNA were confirmed by defining the ratio of absorbance at 260 and 280 nm wavelengths (NanoDrop® ND-1000, ThermoScientific) 700 ng of RNA are transcript into cDNA by using the Transcriptor reverse transcriptase (Roche) and following the manufacturer's instructions. For KIF2A, RT-qPCR reaction were carried out using primers indicated in the sequences (bold and underlined sequences) presented in the table below and with SYBR green I master (Roche) in a Light cycle 480 system. Reaction conditions were carried out for 50 cycles (10 min Initial denaturation 95° C., 10 s at 95° C., 15 s at 58° and 20 s at 72° C.).

TABLE 1
Primer sequences

| Primer | Primer Sequence (5′-3′) |
|---|---|
| KIF2A-E10F | Ccttcgatgactcagctcct |
| | (SEQ ID NO: 15) |
| KIF2A-EnormalF | aatatttgaaaggggcatgg |
| | (SEQ ID NO: 16) |
| KIF2A-EnormalR | ttttcccacttccagtctgc |
| | (SEQ ID NO: 17) |
| KIF2A-E13R | agtaagtcaaacacctttccact |
| | (SEQ ID NO: 18) |
| KIF2A-EdégénérerF | gttggtcgagaccatcttcg |
| | (SEQ ID NO: 19) |
| KIF2A-EdégénérerR | ttgtccgtacgcgaaacag |
| | (SEQ ID NO: 20) |

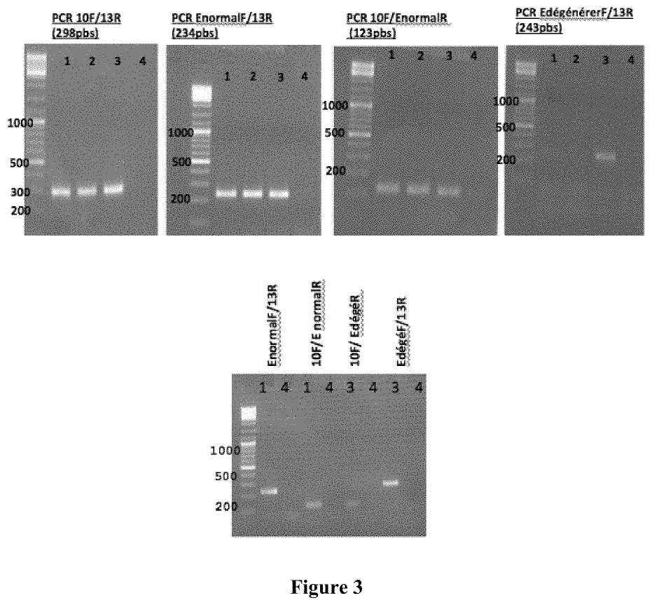

As illustrated in FIG. 3, in the absence of Cre-recombinase, RT-PCR products analyzed either by agarose gel electrophoresis or by sequencing indicate that heterozygous recombinant ES clones express a unique transcript isoform, while after Cre-recombinase action, both alleles are expressed.

Expression of Kif2a in ES cell clones containing the construction prior to and after Cre expression was also analyzed by qRT-PCR. The value of the comparative threshold cycle (ct) of actin and rplPO gene was used as reference and the relative transcript expression of mRNA levels was calculated by the Ct method and by the area of the peak for Kif2a.

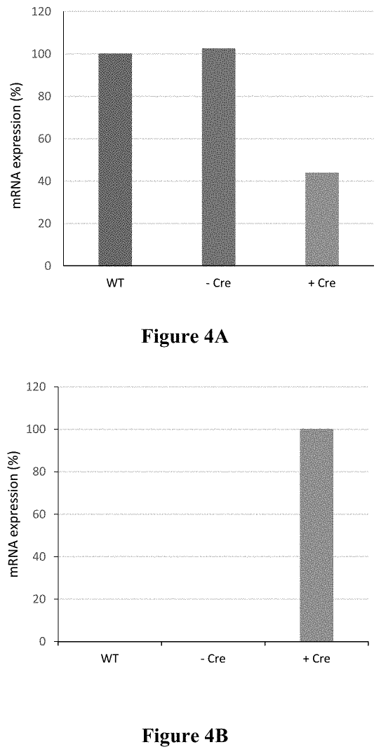

As illustrated in FIG. 4, in the absence of Cre-recombinase the level of expression of WT Kif2a mRNA is comparable to the level of expression of Kif2a in ES control cells; indicating that both alleles are expressed in recombinant ES cells (FIG. 4A). However, after the expression of Cre-recombinase we found that: (i) the level of expression of the WT allele represents only 50% of the one expressed in control ES cells (FIG. 4A), (ii) the mutant allele could specifically be amplified using primers specific to the degenerated sequences (FIG. 4B), (iii) both WT and mutant alleles are expressed at similar levels as evaluated by measuring area under the peaks corresponding to the two alleles (data not shown).

These results confirm the correct expression of WT Kif2a mRNA in ES cells model before the action of the Cre, and the correct switch between WT Kif2a exon and mutant Kif2a degenerated exon after Cre-recombinase expression.

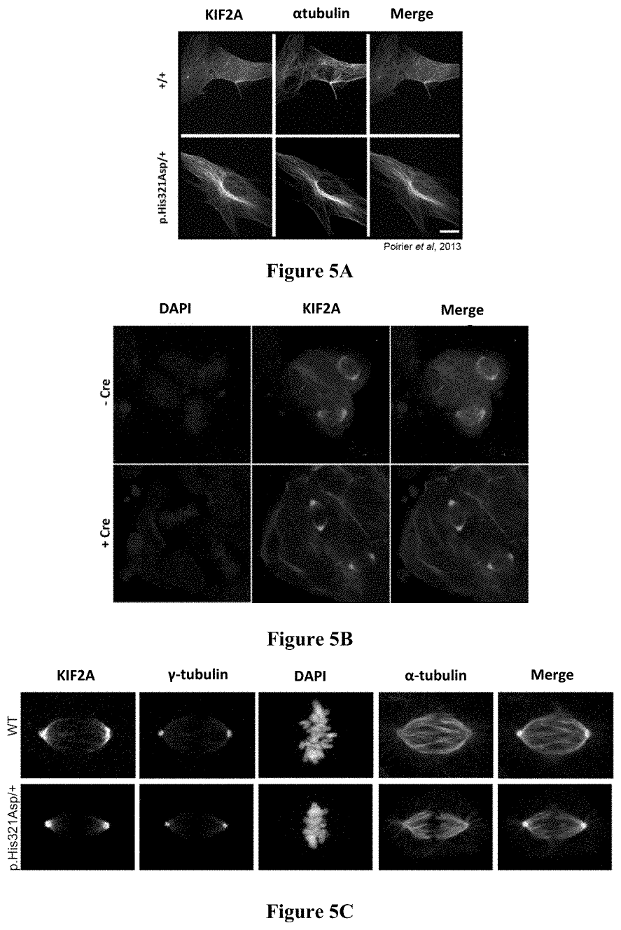

Furthermore, we performed immunofluorescence staining in these ES cell clones to assess KIF2A distribution and whether distribution of mutant KIF2A mimics that the one found the same phenotype showed in the patient fibroblasts bearing c.961C>G, p.His321Asp mutation. In control cells, KIF2A have a diffuse punctiform cytoplasmic and nuclear localization. Patient fibroblasts exhibit a segregation of the KIF2A protein to the microtubules illustrated by a strong colocalization of both of them (FIG. 5A.).

ES cells from the model before the expression of the cre showed the same distribution found in the control fibroblasts. After action of the cre recombinase, ES cells showed the same phenotype in patient fibroblasts with a segregation of the protein in the microtubules. (FIG. 5C.). Moreover, we showed by immunofluorescent staining of control and patient fibroblasts during metaphasis that the Kif2a mutation provoke a reduction of the spindle length and width (FIG. 5B). In the case of the ES cells, we have found the same phenotype, with smaller and thinner spindle after the expression of the Cre than before (FIG. 5C.)

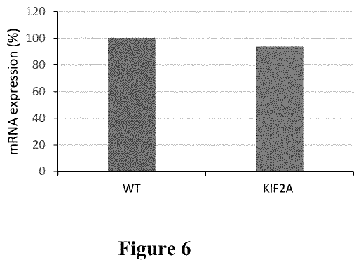

To confirm these results, we performed RT-qPCR on mouse model brain before the expression of the Cre recombinase and WT mouse. We observed that there is the same level of expression of WT Kif2a between the WT mouse and the Kif2a mouse model (FIG. 6). We can therefore conclude that we had a correct mRNA expression of WT KIF2A in the mouse model before the action of the Cre. Altogether, these results confirm an absence of an impact of the construction on the expression of the WT mRNA in both ES cells and mouse model and the correct and expression of the mutant allele switch upon Cre-recombinase expression.

1-19. (canceled)

20. A conditional knock-in cassette which is a double stranded DNA molecule comprising a sequence A, a sequence B, a first pair RTS1 and RTS1′ and a second pair RTS2 and RTS2′ of recombinase target sites (RTS), wherein
(i) the RTS of the first pair and the RTS of the second pair are unable to recombine together, and
(ii) RTS1 and RTS1′ are in an opposite orientation, and
(iii) RTS2 and RTS2′ are in an opposite orientation, and
(iv) sequences A and B and the RTS are in the following order from 5′ to 3′: RTS1, sequence A, RTS2, sequence B, RTS1′ and RTS2′, and
(v) sequences A and B each comprise at least one coding sequence and said coding sequences are on different DNA strands, and
(vi) the amino acid sequence encoded by sequence A has at least 90% sequence identity to the amino acid sequence encoded by sequence B, and
(vii) the coding strand of sequence A and the non-coding strand of sequence B are unable to hybridize.

21. The conditional knock-in cassette of claim 20, wherein the RTS are recognized by the same recombinase.

22. The conditional knock-in cassette of claim 20, wherein the RTS are recognized by a recombinase selected from the group consisting of the Cre recombinase of bacteriophage P1, the FLP recombinase of Saccharomyces cerevisiae, the R recombinase of Zygosaccharomyces rouxii pSR1, the A recombinase of Kluyveromyces drosophilarium pKD1, the A recombinase of Kluyveromyces waltii pKW1, the integrase X Int, the integrase λ Int, the Gin recombinase of the phage Mu, PhiC31 integrase, the Tn3 resolvase, the Dre recombinase, the Tre recombinase, the prokaryotic beta-recombinase, and variants thereof.

23. The conditional knock-in cassette of claim 20, wherein the RTS are recognized by a recombinase selected from the group consisting of the Cre recombinase of bacteriophage P1 and the FLP recombinase of Saccharomyces cerevisiae, and variants thereof.

24. The conditional knock-in cassette of claim 20, wherein the RTS are recognized by the Cre recombinase or a variant thereof.

25. The conditional knock-in cassette of claim 24, wherein the RTS are selected from the group consisting of LoxP site and mutants thereof.

26. The conditional knock-in cassette of claim 24, wherein RTS1 and RTS1′ are LoxP sites and RTS2 and RTS2′ are Lox 511 sites, or vice-versa.

27. The conditional knock-in cassette of claim 20, wherein said at least one coding sequence of sequence A and/or sequence B is an exon or a fragment thereof.

28. The conditional knock-in cassette of claim 20, wherein the amino acid sequence encoded by sequence A has at least 95% sequence identity to the amino acid sequence encoded by sequence B.

29. The conditional knock-in cassette of claim 20, wherein the amino acid sequence encoded by sequence A differs from the amino acid sequence encoded by sequence B by only one amino acid.

30. The conditional knock-in cassette of claim 20, wherein the coding strand of sequence A has less than 60% sequence identity to the coding strand of sequence B.

31. The conditional knock-in cassette of claim 20, wherein the coding sequence(s) of sequence A has (have) less than 70% identity to the coding sequence(s) of sequence B, and the non-coding sequence(s) of sequence A has (have) less than 30%, identity to the non-coding sequence(s) of sequence B.

32. The conditional knock-in cassette of claim 20, wherein the pre-mRNA obtained from the conditional knock-in cassette has a frequency of the minimum free energy RNA secondary structure of 0 and/or an ensemble free energy higher than −800 kcal/mol.

33. The conditional knock-in cassette of claim 20, which further comprises an additional coding sequence.

34. A vector comprising a conditional knock-in cassette as defined in claim 20.

35. An isolated transgenic host cell, excluding a human embryonic cell, comprising a conditional knock-in cassette as defined in claim 20 or a vector comprising said cassette.

36. A non-human transgenic organism comprising at least one cell as defined in claim 35.

37. The transgenic organism of claim 36, which is a mouse.

38. A method of generating a conditional knock-in allele of a target gene in a cell, the method comprising
introducing into the cell a conditional knock-in cassette of claim 20 or a vector comprising said cassette, and
obtaining a transgenic cell in which the conditional knock-in cassette is inserted by homologous recombination into the genome.
